(19) United States
(12) Patent Application Publication
(10) Pub. No.: US 2024/0066470 A1
(43) Pub. Date: Feb. 29, 2024

(54) FILTRATION MEDIA

(71) Applicant: ExThera Medical Corporation, Martinez, CA (US)

(72) Inventors: Robert S. Ward, Martinez, CA (US); Keith R. McCrea, Martinez, CA (US)

(21) Appl. No.: 18/470,986

(22) Filed: Sep. 20, 2023

Related U.S. Application Data
(63) Continuation of application No. PCT/US2022/022748, filed on Mar. 31, 2022.
(60) Provisional application No. 63/171,476, filed on Apr. 6, 2021.

(51) Int. Cl. B01D 61/24; B01D 69/02; B01D 69/08; B01D 71/76

(52) U.S. Cl. CPC B01D 61/243; B01D 69/02; B01D 69/08; B01D 71/76

(57) ABSTRACT

The present disclosure provides a filtration media comprising a combination of (i) a hollow fiber membrane and (ii) adsorption media. The disclosure further provides a device, the device comprising a filtration media, which filtration media includes a combination of (i) a hollow fiber membrane and (ii) adsorption media.

### CROSS-REFERENCE TO RELATED APPLICATION

This present application is a continuation of International Patent Application No. PCT/US2022/022748, filed Mar. 31, 2022, which claims priority to U.S. Provisional Application No. 63/171,476, filed Apr. 6, 2021, the disclosures of which are hereby incorporated by reference in their entirety for all purposes.

### BACKGROUND

Seraph® 100 from ExThera Medical is a CE-Marked device for the reduction of pathogens during bloodstream infections. Treatment with Seraph® 100 can eliminate more than 34 to 99 percent of the pathogens in the incoming patient blood for every pass through the extracorporeal filter. At typical blood flowrates used in chronic dialysis and continuous renal replacement therapy, this produces a large decrease in the patient's bloodstream in just a few hours of treatment.

More recently, Seraph® 100 has been shown to improve hemodynamic stability in coronavirus disease 2019 in those cases requiring mechanical ventilation and vasopressor support. In the treatment of COVID-19 vasopressor dose, body temperature, interleukin-6, and C-reactive protein levels declined after Seraph® 100 treatments. Severe acute respiratory syndrome coronavirus 2 (SARS-CoV-2) viremia was confirmed in a patient tested and cleared by the completion of treatments. (See, SW Olson et al., Crit Care Explor, (8); 2020 August). Due to the encouraging treatment of COVID-19 patients, Seraph® 100 has been granted Emergency Use Authorization (EUA) by the FDA for treatment of respiratory-impacted COVID-19 patients.

Despite the advancements made to date, there is a need for new filtration media for treating patients with acute kidney injury (AKI) or patients suffering from bloodstream infections or other inflammatory states. The current disclosure satisfies these needs and offers other advantages as well.

### BRIEF SUMMARY

In one embodiment, the present disclosure provides a filtration media comprising a combination of (i) a hollow fiber membrane(s) and (ii) adsorption media.

In another embodiment, the disclosure provides a device, the device comprising a filtration media, which filtration media includes a combination of (i) a hollow fiber membrane(s) and (ii) adsorption media.

In certain instances, the combination of (i) a hollow fiber membrane(s) and (ii) adsorption media lowers the amount of a patient's blood volume passed through an extracorporeal device, which improves patient safety. In addition, by placing adsorption media such as heparinized media after or downstream of hollow fiber membranes within a dialyzer, any inflammation caused by the dialyzer (e.g., cell activation or release of inflammatory mediators) is reduced or eliminated. Moreover, by combining (i) a hollow fiber membrane(s) and (ii) adsorption media in a single housing or device, the total amount of housing surface area that a patient's blood is exposed to will decrease, potentially reducing any foreign body immune response. Further, the combination of technologies into one device, requires less tubing, which additionally reduces blood volume and exposure to the surface area of the tubing. Finally, by combining both technologies into one device, priming becomes simplified as instead of the need to properly de-air two devices, only one device needs to be de-aired. This simplifies setup and reduces risk of air embolisms.

These and other aspects, objects and embodiments will become more apparent when read with the detailed description and figures that follow.

### BRIEF DESCRIPTION OF THE DRAWINGS

FIG. 1 illustrates an embodiment of the invention.

FIG. 2 illustrates an embodiment of the invention.

### DETAILED DESCRIPTION

A blood circuit for extracorporeal circulation generally comprises an arterial blood circuit having a dual- or multi-lumen needle or an arterial side puncture needle to draw blood from a patient, a venous blood circuit having a venous side puncture needle on its tip to return the blood to the patient, and a filter between the arterial blood circuit and the venous blood circuit.

In one embodiment, the present disclosure provides a filtration media comprising a combination of (i) a hollow fiber membrane(s) and (ii) adsorption media. The filtration media, methods and devices of the present disclosure are used in blood purification techniques such as those used in hemodialysis and blood filtration. For example, a blood circuit composed of flexible tubes and filtration apparatuses are used so as to extracorporeally circulate a subject's blood through the filtration media as disclosed herein.

A blood circuit having the filtration media of this disclosure can be used in hemodialysis, hemofiltration, or continuous or intermittent renal replacement therapy (RRT). The device can also be used in a blood bank context, where the blood is stored and banked.

In certain aspects, the arterial blood circuit, the filtration media, and the venous blood circuit are connected via flexible tubes, and the blood that is removed from a patient is brought into contact with the device and filtration media via the flexible tubes.

The subject's blood is removed from inside to outside the body of the subject via the arterial blood circuit and then introduced into the blood circuit. The blood then flows through the flexible tubes in the blood circuit to the device(s) comprising the separation media.

In one embodiment, the disclosure provides a filtration media comprising a combination of (i) a hollow fiber membrane(s) and (ii) adsorption media. The blood that is discharged from the filtration media after having passed through the media is returned from the outside to the inside of the subject's body via, for example, the venous blood circuit as blood having a reduced concentration of uremic substances, pathogens, inflammatory molecules, pathogen-associated molecular patterns (PAMPs), damage associated molecular patterns (DAMPs), toxins, protein bound uremic toxins, other sepsis mediators and/or circulating tumor cells (CTCs).

The filtration media of the present disclosure is useful for reducing uremic substances, pathogens, and inflammatory molecules and can be applied to a patient with acute or chronic kidney injury (AKI), whereby it can reduce the concentration of uremic toxins and/or inflammatory molecules in blood of the patient with AKI or chronic kidney disease.

The filtration media of the present disclosure often comprises (i) a hollow fiber membrane(s) and (ii) adsorption media, which allows blood to pass, but adsorbs the toxins, pathogens and other harmful substances. The hollow fiber membrane can be a plurality of hollow fiber membranes.

In certain aspects, the hollow fiber membrane is made of a polymer. Suitable polymers include, but are not limited to, polypropylene, polyethyleneimine-treated polyacrylonitrile (AN69), poly methyl methacrylate (PMMA), polysulfone (PSf), polyethersulfone (PES), polyvinylpyrrolidone (PVP), cellulose triacetate (CA), polyacrylonitrile (PAN), ethylene vinyl alcohol (EVOH), polyvinylidene fluoride (PVDF) and a combination thereof. The hollow fiber membranes are used in artificial dialysis, blood filtration, plasma separation, and the like.

The hollow fiber membrane can be blood-compatible. In certain aspects, the hollow fiber membrane comprises a polymer composed of polysulfone or a mixture of polysulfone and another polymer such as polyvinylpyrrolidone. The polymer combinations can be spun together, or they can be used to coat a hollow fiber membrane.

In certain aspects, the thickness of the hollow fiber membrane is about 0.01 mm to about 5 mm, such as about 0.1 mm, 0.2 mm, 0.3 mm, 0.4 mm, 0.5 mm, 0.6 mm, 0.7 mm, 0.8 mm, 0.9 mm, 1 mm, 1.1 mm, 1.2 mm, 1.3 mm, 1.4 mm, 1.5 mm, 1.6 mm, 1.7 mm, 1.8 mm, 1.9 mm, 2 mm, 2.1 mm, 2.2 mm, 2.3 mm, 2.4 mm, 2.5 mm, 2.6 mm, 2.7 mm, 2.8 mm, 2.9 mm, 3 mm, 3.1 mm, 3.2 mm, 3.3 mm, 3.4 mm, 3.5 mm, 3.6 mm, 3.7 mm, 3.8 mm, 3.9 mm, 4 mm, 4.1 mm, 4.2 mm, 4.3 mm, 4.4 mm, 4.5 mm, 4.6 mm, 4.7 mm, 4.8 mm, 4.9 mm, and/or 5 mm. In certain aspects, the hollow fiber membrane is used as a hollow fiber membrane-type module inserted into a housing, container, or device.

In certain aspects, the hollow fiber membrane is contained in a housing, such as for example, a bundle of hollow fiber membranes composed of numerous short to medium lengths of hollow fiber membranes inserted in a cylindrical housing as a hollow fiber membrane column.

In certain aspects, the disclosure provides a filtration media comprising a combination of (i) a hollow fiber membrane and (ii) adsorption media. The (ii) adsorption media is typically a coated substrate. Suitable substrates for the adsorption media include, but are not limited to, non-porous rigid beads including polymer beads, particles, or packing, reticulated foams, a rigid monolithic bed (e.g. formed from sintered beads or particles), a column packed with woven or non-woven fabric, a column packed with a yarn or solid or hollow dense (not microporous) monofilament fibers, a spiral wound cartridge formed from flat film or dense membrane, or a combination of media such as a mixed bead/fabric cartridge. A suitable substrate for use in the present disclosure is one that is initially microporous, but becomes essentially nonporous when the surface is treated before, during or after the creation of adsorption sites, e.g., coated with a polysaccharide such as end-point-attached heparin.

In certain aspects, the adsorption media has a macroporous structure that presents a high surface area to the blood or serum, while preventing a large pressure drop and high shear rates. In addition to the potential for damaging the blood by hemolysis, high pressure drops should be avoided because they can shut down extracorporeal circuits equipped with automatic shut offs that respond to pressure drop. The substrate may also take the form of a dense barrier membrane, in a spiral wound configuration, for example. In certain aspects, the surface of a non-porous film is modified by a coating such as by binding heparin, heparan sulphate or another adsorbent polysaccharide together with optional adsorbing groups not derived from heparin, heparan sulphate, or the adsorbent polysaccharide to the membrane's surface.

In certain aspects, adsorption media is a surface coated solid substrate. Suitable surface coatings include heparin, polyethyleneimine (PEI), sialic acid, hyaluronic acid, polyvinylpyrrolidone (PVP), and combination thereof.

In certain other aspects, the surface coatings include, but are not limited to, a monoclonal antibody, a protein, a carbohydrate, a polysaccharide and a combination thereof.

In certain aspects, the surface area of the adsorption media is between about 5 m² and about 50 m², such as between about 10 m² and about 20 m².

In certain aspects, filtration media is provided packed within a container or housing, such as a column, that is designed to hold a combination of a hollow fiber membrane and adsorption media so that it will not be carried away in the flowing blood (‘preventing media migration’), and permit the flow of blood past essentially all of the media's surface.

In certain aspects, the adsorption media are beads.

In certain aspects, the adsorption media substrate is in the form of solid beads or particles. The ‘beads’ can be made of materials that are sufficiently rigid to resist deformation/compaction under the encountered flow rates and pressures (such as polymer beads). Resistance to deformation is an advantage to maintain the interstitial dimensions and overall free volume and subsequent low pressure drop of the packed bed contactor. The dimensional stability of the packed bed is also important in maintaining sufficient inter-bead separation to avoid filtering out blood cells. The substantial lack of accessible pores in the bulk of the substrate eliminates the need for adsorbates to diffuse into the pores prior to adsorption/binding. The adsorption sites of the present disclosure are primarily on the surface of the media and are thus positioned to be accessible to adsorbates in the blood delivered to that surface largely by convective transport. Suitable substrates need not be perfectly smooth on their surface since roughness produces a desirable increase in surface area for attachment of binding sites, e.g., by ionic or preferably covalent bonding of heparin. Accessible internal pores with molecular dimension, on the other hand, are largely avoided to eliminate the need for adsorbates to diffuse into the pores before attaching to binding sites.

Various kinds of beads can be used in the present disclosure. Suitable beads have sufficient size and rigidity to avoid deformation/compaction during use in the method and have sufficient surface area to be capable of being coated with heparin for use in the method.

The beads or other high-surface-area substrates may be made from a number of different biocompatible materials, such as natural or synthetic polymers or non-polymeric material including glasses, ceramics and metals, that are essentially free of leachable impurities. Some exemplary polymers including polyurethane, polymethylmethacrylate, polyethylene or co-polymers of ethylene and other monomers, polyethylene imine, polypropylene, and polyisobutylene. Examples of useful substrates include nonporous Ultra High Molecular Weight PolyEthylene (UHMWPE). Other suitable beads are optionally cross-linked polystyrene, high density and low density polyethylene, silica, polyurea, and chitosan.

Methods for making such beads are known in the art. Polyethylene beads and other polyolefin beads are produced directly during the polymer synthesis process and can often be used without further size reduction. Other polymers may need to be ground or spray dried and classified, or otherwise processed to create beads of the desired size distribution and shape.

In certain aspects, the size of the channels or interstitial space between individual beads for extracorporeal blood filtration reduce or eliminate a high-pressure drop between the inlet and outlet of the cartridge, to permit safe passage of the blood cells between the individual beads in a high flow environment, and to provide appropriate interstitial surface area for binding of the polysaccharide adsorbent to the toxins, cytokines or pathogens in the blood. In a close packed bed of 300-micron, roughly spherical beads, an appropriate interstitial pore size is approximately 68 microns in diameter. Useful beads have a size ranging from about 100 to above 500 microns in diameter such as about 100 microns, 125 microns, 150 microns, 175 microns, 200 microns, 225 microns, 250 microns, 275 microns, 300 microns, 325 microns, 350 microns, 375 microns, 400 microns, 425 microns, 450 microns, 475 microns, and/or 500 microns. The average size of the beads can be from 150 to 450 microns. For example, polyethylene beads from Polymer Technology Group (Berkeley, USA) having an average diameter of 0.3 mm are suitable. The interstitial pore is a function of bead size. For use, the suitable beads are housed in a container, such as a column.

In certain aspects, other suitable forms of substrates include reticulated foams. Reticulated foams have open cells and can be made from, for example, polyurethanes and polyethylenes. Control of pore size can be achieved by controlling the manufacturing method. In general, reticulated foams can have between 3 and 100 pores/inch and can exhibit a surface area of >66 cm².

In certain aspects, beads can be sintered into a monolithic porous structure through either chemical or physical means. Polyethylene beads can be sintered by heating the beads above their melting temperature in a cartridge and applying pressure. The resulting interstitial pore size is slightly reduced from the interstitial pore size of a packed bed of non-sintered beads of equal size. This reduction can be determined empirically and used to produce the desired final interstitial pore size.

In certain aspects, the adsorbent polysaccharide of the adsorbent media can be bound to the surface of the solid substrate (e.g., bead) by various methods, including covalent attachment or ionic attachment. The adsorption media can comprise heparin covalently linked to the surface of the solid substrate. In one embodiment, the heparin is linked to the solid substrate by covalent end-point attachment. This method increases the safety of the device by reducing or eliminating the release of heparin from the substrate surface that could enter the blood stream. Leaching of heparin by and into the blood is to be avoided because it can increase the risk of bleeding and heparin-induced thrombocytopenia. Covalent attachment of the polysaccharide, such as heparin, to a solid substrate provides better control of parameters such as surface density and orientation of the immobilized molecules as compared to non-covalent attachment. These parameters have been shown to be advantageous in order to provide optimal Antithrombin III, cytokine or pathogen binding to the immobilized carbohydrate molecules. The surface concentration of heparin on the solid substrate is often in the range of 1-10 μg/cm². Covalent end-point attachment means that the polysaccharide, such as heparin is covalently attached to the solid substrate via the terminal residue of the heparin molecule. Heparin can also be bound to the surface at multiple points. However, the end-point attachment is preferred.

In certain aspects, the heparin is full length heparin having a mean molecular weight in the range of 15-25 kDa, such as about a mean molecular weight of 21 kDa or more.

In certain aspects, the heparin has a surface concentration of 1-20 μg/cm², such as about 5-15 μg/cm².

In certain aspects, the heparin is full length heparin covalently attached to a solid substrate via stable secondary amino groups.

In certain aspects, a total surface area of the solid substrate is in the range of 0.5-3 m².

In certain aspects, the beads may be hydrophilized prior to attachment of the polysaccharide, such as heparin, or other compounds. Possible methods of preparing the beads include acid etching, plasma treating, and exposure to strong oxidizers such as potassium permanganate.

In certain aspects, the adsorption media is sized to be larger than the inner diameter of the hollow fiber membrane.

In certain other instances, the adsorption media contains materials similar to the filter or filtration device of the Seraph® Microbind® Affinity Blood Filter, which is a filter that allows body fluids to pass over microbeads coated with molecular receptor sites that mimic the receptors on human cells which pathogens use to colonize when they invade the body. The adsorption media is a flexible platform that uses covalently-bonded, immobilized heparin or heparan sulfate for its unique binding capacity. See, for example, U.S. Pat. Nos. 8,663,148, 8,758,286 or 9,173,989, disclosing at least one polysaccharide adsorbent, or immobilized heparin, each of which is incorporated by reference.

In certain other instances, the adsorption media can be for example, similar material as the extracorporeal hemoadsorption filter device to remove cytokines from circulating blood such as a biocompatible, sorbent bead technology e.g., CytoSorb™, CytoSorbents™ Inc. CytoSorb hemoadsorption beads are polystyrene-divinylbenzene porous particles (450 μm avg. particle diameter, 0.8-5 nm pore diameter, 850 m²/g surface area) with a biocompatible polyvinyl-pyrrolidone coating. See for example, U.S. Pat. No. 8,647,666 which claims a method of using a composition comprising polystyrene divinyl benzene copolymer and a polyvinyl pyrrolidone polymer.

In one embodiment, the disclosure provides a device, the device comprising a filtration media, which filtration media includes a combination of (i) a hollow fiber membrane and (ii) adsorption media.

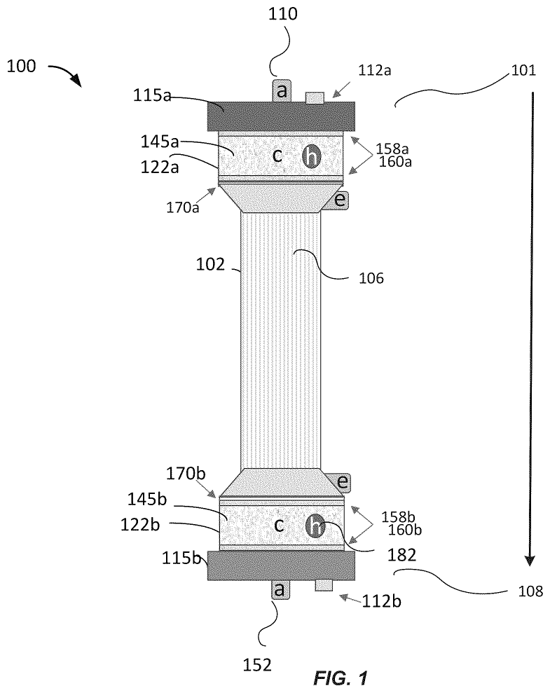

Turning now to FIG. 1, as shown therein, one embodiment of a device 100 of the present disclosure can be a column, housing or container containing or holding a filtration media, which filtration media includes a combination of a hollow fiber membrane and adsorption media. In this embodiment, the device 100 has a first end 101, a substantially cylindrical portion 102 housing the hollow fiber membrane 106 and a second end 108. During blood flow through the device 100, whole blood or body fluid(s) enters the device 100 through a connection or adapter 110 such as a luer connection. The body fluid can flow through this device clockwise or counterclockwise. As shown in FIG. 1, the device has a first end cap 115a and a second end cap 115b. In this configuration, between the first end cap 115a and the cylindrical portion 102, adsorption media 145a is housed in a portion of the device 122a. As shown, the device 100 contains a first adsorption media 145a, a hollow fiber membrane 106 and a second adsorption media 145b, wherein the hollow fiber membrane 106 is sandwiched between the first adsorption media 145a and second adsorption media 145b. The adsorption media 145a and 145b can be the same or different.

The housing or device 100 has an inlet port 110 and an outlet port 152 for a fluid so as to run the fluid (e.g., whole blood or body fluid) through the first adsorption media 145a, the hollow fiber membrane 106 and the second adsorption media 145b (in the direction of the arrow 101 to 108). In certain aspects, the first absorption media 145a or alternatively, the second adsorption media 145b can be optional. In this configuration, 122a or 122b is omitted and the device has only 1 adsorption media portion.

In certain aspects, the first adsorption media 145a and the second adsorption media 145b are the same material, such as heparin coated beads.

In certain aspects, the first adsorption media 145a and the second adsorption media 145b are different material. For example, the beads can have different polysaccharide coatings or different combination of polysaccharides.

In certain aspects, a screen or a retention filter 158a is disposed between the hollow fibers 106 and the adsorption media 145a at the first end 101. Similarly, a screen or a retention filter 158b is disposed between the hollow fibers 106 and the adsorption media 145b at the second end 108.

In certain aspects, the first end cap 115a and the second end cap 115b have an attachment for body fluid ingress 110 and/or egress 152 of the device. In certain aspects, the attachment is a luer fitting. Although body fluid ingress 110 and egress 152 of the device are shown, in an alternative aspect, the body fluid can flow through the device in reverse with fluid ingress 152 and fluid egress 110.

In certain aspects, the first adsorption media 145a is disposed between a pair of retention plates 158a, 160a. Similarly, in certain aspects, the second adsorption media 145b is disposed between a pair of retention plates 158b, 160b.

In certain aspects, the pair of retention plates is a screen or porous substrate.

In certain aspects, the first end cap 115a comprises a vent 112a.

In certain aspects, the second end cap 115b comprises a vent 112b.

In certain aspects, the device has a first end cap 115a and a second end cap 115b the filtration media being disposed therebetween, 145a, 106, 145b. In an alternative embodiment, either 145a or 145b is omitted.

In certain aspects, the device has a gap 170a and 170b between retention plates and potting material.

In certain aspects, the device has a media fill port 182.

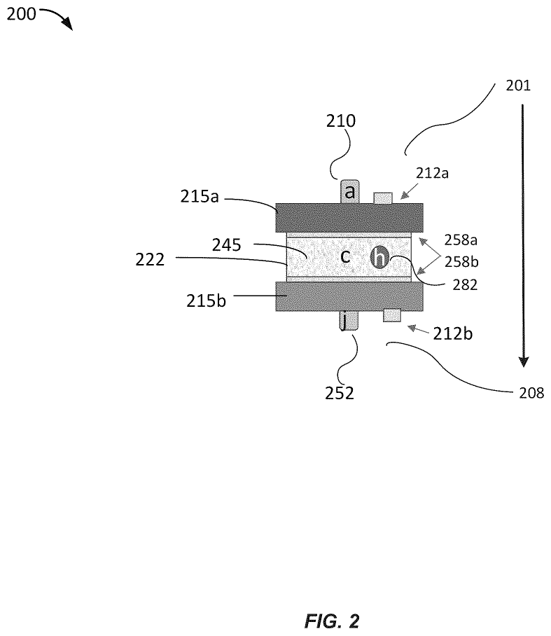

Turning now to FIG. 2, the disclosure provides a device 200 that is fitted directly to a dialyzer. In other words, in certain aspects, when fitted, the device 200 will allow a commercial dialyzer to function similarly to a device of FIG. 1, 100. For example, commercial dialyzers typically contain hollow fiber membranes made of for example, polysulfone (PSf), modified cellulose or ethylene vinyl alcohol copolymer (EVAL). The device 200 can be fitted directly on a dialyzer, either above or below the dialyzer or even both above and below the dialyzer with two devices.

FIG. 2 shows a device 200 of the present disclosure which is a column, housing or container containing adsorption media. In this embodiment, the device 200 has a first end 201, a portion 222 housing the adsorption media 245 and a second end 208. During blood flow through the device 200, whole blood or body fluid enters the device 200 at the first end 201 through a connection or adapter 210 such as a luer connection (e.g., male adaptor). As shown in FIG. 2, the device has a first end cap 215a and a second end cap 215b. In this configuration, between the first end cap 215a and the cylindrical portion 222, absorption media 245 is housed.

In certain aspects, a screen or a retention filter 258a is disposed between the adsorption media 245 at the first end 201. Similarly, a screen or a retention filter 258b is disposed between the adsorption media 245 at the second end 208.

In certain aspects, the first end cap 215a and the second end cap 215b have an attachment for body fluid ingress 210 and/or egress 252 of the device. In certain aspects, the attachment is a luer fitting. Although body fluid ingress 210 and egress 252 of the device are shown, in an alternative aspect, the body fluid can flow through the device in reverse with fluid ingress 252 and fluid egress 210.

In certain aspects, the adsorption media 245 is disposed between a pair of retention plates 258a, 258b.

In certain aspects, the pair of retention plates is a screen or porous substrate.

In certain aspects, the first end cap 215a comprises a vent 212a.

In certain aspects, the second end cap 215b comprises a vent 212b.

In certain aspects, the device optionally has a media fill port 282.

In certain aspects, the device is used for a therapy which is a member selected from the group consisting of hemodialysis, hemofiltration, renal replacement therapy (RRT), hemoperfusion, and/or glycocalyx replacement therapy.

In addition to a dialyzer, the device and methods of the present disclosure can be used in combination with other extracorporeal organ support devices (ECOS) such as extracorporeal membrane oxygenation, extracorporeal CO₂ removal [ECCO₂R] and extracorporeal liver support.

In certain aspects, the device is used for acute kidney injury (AKI) or patients suffering from bloodstream infections or other inflammatory states.

In certain aspects, the hollow fibers remove renal toxins while the adsorption media removes pathogens, inflammatory molecules, pathogen-associated molecular patterns (PAMPs), damage associated molecular patterns (DAMPs), toxins, circulating tumor cells (CTCs), protein bound uremic toxins, and other sepsis mediators.

### EXAMPLES

### 1. Comparative Example 1

A dialysis blood circuit is constructed with a commercial dialyzer. Both the blood and dialysate sides of the dialyzer are primed with 500 ml of phosphate buffered saline (PBS) at a flow rate of 200 ml/min. Thereafter a test sample of mammalian blood comprising urea and methicillin-resistant Staphylococcus aureus (MRSA) bacteria is passed through the system. A significant amount of the urea can be removed by the dialyzer; however, the MRSA remains in the blood. After several passes of the blood sample through the blood circuit, inflammatory mediators such as IL-1β, TNF-α, IL-6 and IL-15 and chemokines such as IL-8 and GRO-α show an increase in concentration in the blood sample.

### 2. Comparative Example 2

A dialysis blood circuit is constructed with a Seraph® Microbind® Affinity Blood Filter. Both the blood and dialysate sides of the blood filter device comprising Seraph® are primed with 500 ml of PBS at a flow rate of 200 ml/min. Thereafter a test sample of mammalian blood comprising urea and methicillin-resistant Staphylococcus aureus (MRSA) bacteria is passed through the system. Only MRSA is removed from the sample, the urea remains in the blood sample.

### 3. Experimental Example 1

A dialysis blood circuit is constructed with a commercial dialyzer and a Seraph® Microbind® Affinity Blood Filter. Both the blood and dialysate sides of the combination of dialyzer and filter are primed with 500 ml of PBS at a flow rate of 200 ml/min. A significant amount of the urea is removed by the dialyzer, and the MRSA is removed by the Seraph® filter. After several passes of the blood sample through the blood circuit, no inflammatory mediators are present in the blood sample.

### 4. Comparative Example 3

In a first blood circuit, the blood circuit has 2 devices in series (i) a Seraph® Microbind® Affinity Blood Filter and (ii) a commercial dialyzer device used in combination. In a second blood circuit, an inventive device is used as a single device which is a combination of a Seraph® Microbind® Affinity Blood Filter and a dialyzer (a hollow fiber membrane). By combining the technology of two columns (circuit 1) into the inventive second blood circuit, (Seraph® Microbind® Affinity Blood Filter+a dialyzer hollow fiber membrane) into a single column several advantages are realized. The inventive single column reduces blood volume by removing the need for two additional column endcaps. Each column, alone, has two endcaps, which takes up volume and running two columns in series in the first blood circuit means there are 4 endcaps. The inventive hybrid device only needs two endcaps, and therefore the total blood volume becomes less.

For example, as an estimate, the total blood volume in blood circuit 1 (Seraph® Microbind® Affinity Blood Filter+dialyzer) is estimated to about 250 mL. The blood volume of each one of the Seraph® endcaps is about 25 mL and dialyzer's endcaps is about 5 mL. Therefore, combining all endcaps together in blood circuit 1, is estimated to be about 60 mL of blood needed to fill that volume during use.

In contrast, the inventive hybrid device is estimated to be about 30 mL due to 2 less encaps (one less from Seraph® and one less from the dialyzer). This results in a safer device as less blood is removed from the patient during a treatment. Less blood may result in fewer hypotension events, which often occur at the start of extracorporeal sessions due to the ‘loss’ of blood during the first few minutes of the treatment.

Although the Seraph® blood filter can be positioned upstream or downstream of the dialyzer, the use of Seraph® Microbind® Affinity Blood Filter just upstream of the dialyzer acts as a depth filter that prevents micro-clots from entering and blocking the hollow fiber membranes of the dialyzer while also presenting an anti-thrombogenic surface that may also act to prevent clotting within the dialyzer. This in turn will keep the dialyzer operating at maximum efficiency and also prevent alarms that requires a healthcare worker's involvement. Safety is enhanced via regular or intermittent prophylactic use of a small Seraph cartridge to remove pathogens thereby preventing serious bloodstream infections/sepsis, the second leading cause of death among chronic dialysis patients.

### EMBODIMENTS

Embodiment 1. A filtration media comprising a combination of a hollow fiber membrane and adsorption media.

Embodiment 2. The filtration media of embodiment 1, wherein the hollow fiber membrane is made of a polymer.

Embodiment 3. The filtration media of embodiment 2, wherein the polymer is a member selected from the group consisting of polypropylene, polyethyleneimine-treated polyacrylonitrile (AN69), poly methyl methacrylate (PMMA), polysulfone (PSf), polyethersulfone (PES), cellulose triacetate (CA), polyacrylonitrile (PAN), ethylene vinyl alcohol (EVOH), polyvinylidene fluoride (PVDF) and a combination thereof.

Embodiment 4. The filtration media of any one of embodiments 1-3, wherein adsorption media is a surface coated solid substrate.

Embodiment 5. The filtration media of embodiment 4, wherein the surface. coating is a member selected from the group consisting of heparin, polyethyleneimine (PEI), sialic acid, hyaluronic acid, polyvinylpyrrolidone (PVP), and combination thereof.

Embodiment 6. The filtration media of any one of embodiments 1-5, wherein the surface coating is a member selected from the group consisting of a monoclonal antibody, a protein, a carbohydrate and a combination thereof.

Embodiment 7. The filtration media of any one of embodiments 1-6, wherein the surface area of the adsorption media is between about 5 m² and about 50 m².

Embodiment 8. The filtration media of any one of embodiments 1-7, wherein the surface area of the adsorption media is between about 10 m² and about 20 m².

Embodiment 9. The filtration media of any one of embodiments 1-8, wherein the adsorption media is sized to be larger than the inner diameter of the hollow fiber membrane.

Embodiment 10. The filtration media of any one of embodiments 1-9, wherein the adsorption media are beads.

Embodiment 11. A device, the device comprising a filtration media, which filtration media includes a combination of (i) a hollow fiber membrane and (ii) adsorption media.

Embodiment 12. The device of embodiment 11, wherein the device has a first end cap and a second end cap the filtration media being disposed therebetween.

Embodiment 13. The device of embodiment 11, wherein the filtration media is in a sandwich configuration of a first adsorption media portion, a hollow fiber membrane portion and a second adsorption media portion, wherein the hollow fiber membrane is sandwiched between the first and second adsorption media portions.

Embodiment 14. The device of embodiment 13, wherein the first adsorption media and the second adsorption media are the same material.

Embodiment 15. The device of embodiment 13, wherein the first adsorption media and the second adsorption media are different material.

Embodiment 16. The device of any one of embodiments 11-15, wherein the hollow fiber membrane is made of a polymer.

Embodiment 17. The device of any one of embodiments 11-16, wherein the polymer is a member selected from the group consisting of polypropylene, polyethyleneimine-treated polyacrylonitrile (AN69), poly methyl methacrylate (PMMA), polysulfone (PSf), polyethersulfone (PES), cellulose triacetate (CA), polyacrylonitrile (PAN), ethylene vinyl alcohol (EVOH), polyvinylidene fluoride (PVDF) and combination thereof.

Embodiment 18. The device of any one of embodiments 11-17, wherein adsorption media is a surface coated solid substrate.

Embodiment 19. The device of embodiment 18, wherein the surface coating is a member selected from the group consisting of heparin, polyethyleneimine (PEI), sialic acid, hyaluronic acid, polyvinylpyrrolidone (PVP), and combination thereof.

Embodiment 20. The device of any one of embodiments 18-19, wherein the surface coating is a member selected from the group consisting of a monoclonal antibody, a protein, a carbohydrate and combinations thereof.

Embodiment 21. The device of any one of embodiments 11-20, wherein the surface area of the adsorption media is between about 5 m² and about 50 m².

Embodiment 22. The device of any one of embodiments 11-21, wherein the surface area of the adsorption media is between about 10 m² and about 20 m².

Embodiment 23. The device of any one of embodiments 11-22, wherein the adsorption media is sized to be larger than the inner diameter of the hollow fiber membrane.

Embodiment 24. The device of any one of embodiments 11-23, wherein a screen or a retention filter is disposed between the hollow fibers and the adsorption media.

Embodiment 25. The device of any one of embodiments 12-24, wherein the first end cap and the second end cap have an attachment for body fluid ingress and/or egress of the device.

Embodiment 26. The device of embodiment 25, wherein the attachment is a luer fitting.

Embodiment 27. The device of any one of embodiments 13-26, wherein the first adsorption media portion is disposed between a pair of retention plates.

Embodiment 28. The device of embodiment 27, wherein the pair of retention plates is a screen or porous substrate.

Embodiment 29. The device of any one of embodiments 13-26, wherein the second adsorption media portion is disposed between a pair of retention plates.

Embodiment 30. The device of embodiment 29, wherein the pair of retention plates is a screen or porous substrate.

Embodiment 31. The device of any one of embodiments 12-28, wherein the first end cap comprises a vent.

Embodiment 32. The device of any one of embodiments 12-31, wherein the second end cap comprises a vent.

Embodiment 33. The device of any one of embodiments 11-32, wherein the device is fitted to a dialyzer or other organ support device.

Embodiment 34. The device of any one of embodiments 11-33, wherein the device is used for a therapy which is a member selected from the group consisting of hemodialysis, hemofiltration, renal replacement therapy (RRT), hemoperfusion, and/or glycocalyx replacement therapy.

Embodiment 35. The device of any one of embodiments 11-33, wherein the device is used for acute kidney injury (AKI) or patients suffering from bloodstream infections or other inflammatory states.

Embodiment 36. The device of any one of embodiments 11-33, wherein the hollow fibers remove renal toxins while the adsorption media removes pathogens, inflammatory molecules, pathogen-associated molecular patterns (PAMPs), damage associated molecular patterns (DAMPs), toxins, protein bound uremic toxins, circulating tumor cells (CTCs), and other sepsis mediators.

Embodiment 37. A method for reducing inflammation in a patient while using a dialyzer, the method comprising:

contacting a body fluid post dialysis with an adsorption media, wherein the adsorption media comprises a surface coated solid substrate.

Embodiment 38. The method of embodiment 37, wherein the surface coating is a member selected from the group consisting of heparin, polyethyleneimine (PEI), sialic acid, hyaluronic acid, polyvinylpyrrolidone (PVP), and combination thereof.

Embodiment 39. The method of any one of embodiments 37-38, wherein the surface coating is a member selected from the group consisting of a monoclonal antibody, a protein, a carbohydrate and a combination thereof.

Embodiment 40. The method of any one of embodiments 37-39, wherein the surface area of the adsorption media is between about 5 m² and about 50 m².

Embodiment 41. The method of any one of embodiments 37-40, wherein the surface area of the adsorption media is between about 10 m² and about 20 m².

Embodiment 42. The method of any one of embodiments 37-41, wherein the adsorption media is sized to be larger than the inner diameter any upstream hollow fiber membrane.

One of ordinary skill in the art will readily appreciate that the above description is not exhaustive, and that aspects of the disclosed subject matter may be implemented other than as specifically disclosed above. Indeed, embodiments of the disclosed subject matter can be implemented in hardware and/or software using any known or later developed systems, structures, devices, and/or software by those of ordinary skill in the applicable art from the functional description provided herein.

In this application, unless specifically stated otherwise, the use of the singular includes the plural, and the separate use of “or” and “and” includes the other, i.e., “and/or.” Furthermore, use of the terms “including” or “having,” as well as other forms such as “includes,” “included,” “has,” or “had,” are intended to have the same effect as “comprising” and thus should not be understood as limiting.

Any range described herein will be understood to include the endpoints and all values between the endpoints. Whenever “substantially,” “approximately,” “essentially,” “near,” or similar language is used in combination with a specific value, variations up to and including 10% of that value are intended, unless explicitly stated otherwise.

It is thus apparent that there is provided, in accordance with the present disclosure, extracorporeal blood treatment systems and methods employing batch processing. Many alternatives, modifications, and variations are enabled by the present disclosure. While specific examples have been shown and described in detail to illustrate the application of the principles of the present invention, it will be understood that the invention may be embodied otherwise without departing from such principles. For example, disclosed features may be combined, rearranged, omitted, etc. to produce additional embodiments, while certain disclosed features may sometimes be used to advantage without a corresponding use of other features. Accordingly, Applicant intends to embrace all such alternative, modifications, equivalents, and variations that are within the spirit and scope of the present invention. All references mentioned throughout the disclosure are hereby incorporated by reference.

What is claimed is:

1. A filtration media comprising a combination of a hollow fiber membrane and adsorption media.

2. The filtration media of claim 1, wherein the hollow fiber membrane is made of a polymer.

3. The filtration media of claim 2, wherein the polymer is a member selected from the group consisting of polypropylene, polyethyleneimine-treated polyacrylonitrile (AN69), poly methyl methacrylate (PMMA), polysulfone (PSf), polyethersulfone (PES), cellulose triacetate (CA), polyacrylonitrile (PAN), ethylene vinyl alcohol (EVOH), polyvinylidene fluoride (PVDF) and a combination thereof.

4. The filtration media of claim 1, wherein adsorption media is a surface coated solid substrate.

5. The filtration media of claim 4, wherein the surface coating is a member selected from the group consisting of heparin, polyethyleneimine (PEI), sialic acid, hyaluronic acid, polyvinylpyrrolidone (PVP), and combination thereof.

6. The filtration media of claim 1, wherein the surface coating is a member selected from the group consisting of a monoclonal antibody, a protein, a carbohydrate and a combination thereof.

7. The filtration media of claim 1, wherein the surface area of the adsorption media is between about 5 m² and about 50 m².

8. The filtration media of claim 1, wherein the surface area of the adsorption media is between about 10 m² and about 20 m².

9. The filtration media of claim 1, wherein the adsorption media is sized to be larger than the inner diameter of the hollow fiber membrane.

10. The filtration media of claim 1, wherein the adsorption media are beads.

11. A device, the device comprising a filtration media, which filtration media includes a combination of (i) a hollow fiber membrane and (ii) adsorption media.

12. The device of claim 11, wherein the device has a first end cap and a second end cap the filtration media being disposed therebetween.

13. The device of claim 11, wherein the filtration media is in a sandwich configuration of a first adsorption media portion, a hollow fiber membrane portion and a second adsorption media portion, wherein the hollow fiber membrane is sandwiched between the first and second adsorption media portions.

14. The device of claim 13, wherein the first adsorption media and the second adsorption media are the same material.

15. The device of claim 13, wherein the first adsorption media and the second adsorption media are different material.

16. The device of claim 11, wherein the hollow fiber membrane is made of a polymer.

17. The device of claim 11, wherein the polymer is a member selected from the group consisting of polypropylene, polyethyleneimine-treated polyacrylonitrile (AN69), poly methyl methacrylate (PMMA), polysulfone (PSf), polyethersulfone (PES), cellulose triacetate (CA), polyacrylonitrile (PAN), ethylene vinyl alcohol (EVOH), polyvinylidene fluoride (PVDF) and combination thereof.

18. The device of claim 11, wherein adsorption media is a surface coated solid substrate.

19. The device of claim 18, wherein the surface coating is a member selected from the group consisting of heparin, polyethyleneimine (PEI), sialic acid, hyaluronic acid, polyvinylpyrrolidone (PVP), and combination thereof.

20. A method for reducing inflammation in a patient while using a dialyzer, the method comprising:
contacting a body fluid post dialysis with an adsorption media, wherein the adsorption media comprises a surface coated solid substrate.
